(19) United States
(12) Patent Application Publication
(10) Pub. No.: US 2005/0238737 A1
(43) Pub. Date: Oct. 27, 2005

(54) Use of one or more shogaol(s) as an aphrodisiac

(76) Inventors: Daniel Jean, Vic Le Comte (FR); Leon Cariel, Paris (FR)

Correspondence Address: Steptoe & Johnson, 1330 Connecticut Avenue NW, Box PTO, Washington, DC, 20036, US

(21) Appl. No.: 10/473,919

(22) PCT Filed: Mar. 5, 2002
(86) PCT No.: PCT/FR02/00784

§ 371 Date: Sep. 5, 2003

(30) Foreign Application Priority Data
Mar. 5, 2001 (FR) 01/02961

(51) Int. Cl. A61K 31/12; A61K 35/78

(52) U.S. Cl. 424/756; 514/678

(57) ABSTRACT

The present invention relates to the use of one or more shogaol(s) as aphrodisiacs.
Advantageously, the shogaol(s) correspond(s) to the general formula I: 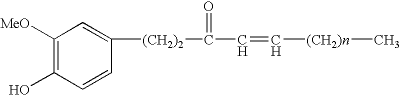 in which n is equal to 1, 2, 4, 6 or 8 and advantageously 1. The present invention also relates to a process for stimulating or arousing the libido in human beings, comprising the administration to a human being of an effective amount of one or more shogaol(s).

The present invention relates to aphrodisiacs. It relates in particular to the use of one or more shogaol(s) as aphrodisiacs.

Since the dawn of time, many substances have been used with the aim of increasing the sexual desire (the libido). Some of them have met with great success and have been handed down through the centuries. However, none of the substances has survived the meticulous scrutiny of scientists. Consequently, the search for a genuine aphrodisiac continues.

The Applicant has found, surprisingly, that shogaol(s) has(have) an aphrodisiac action on men and women.

Shogaols have already been described as having deodorant and/or antiseptic activity (FR 2 758 086).

Certain shogaols are also known chemical constituents of plants of the Alpinia genus, such as Alpinia galanga or Alpinia officinarun or Zingiber, such as Zingiber officinalis, Zingiber cassumunar or Zingiber zerumbet, obtained by extraction of their rhizome.

In particular, the ginger belonging to the Zingiber officinalis family is a well-known spice used in many culinary preparations and has the reputation of being aphrodisiac. However, in view of the quantity of chemical constituents present in its rhizome (geraniol, geranyl acetate, linalool, citronyl acetate, α-terpineol, borneol, bornyl acetate, neral, genarial, β-bisabolene, (−)-zingiberene, (+)-ar-curcumene, β-sesquiphellandrene, gingerols, etc.), it is difficult to know to which of them this aphrodisiac activity is due. Furthermore, although the other pharmaceutical properties of ginger, such as its antiemetic action, its action against travel sickness and its anti-inflammatory action, have been scientifically proven, this has never been so for its aphrodisiac activity.

Similarly, although Alpinia galanga has also been considered in ancient Indian and Arabic manuscripts as having an aphrodisiac effect (Indian Materia Medica K. M., 1976, vol. 1, p. 79, Bombay Popular Prakashan Ed.; Notices et manuscrits de la Bibliothèque Nationale [Notices and Manuscripts of the National Library], Volume 25, IBN EL BEITAR, p. 61, 1883, Editions de l' Institut de France) has this effect, not proven to date, may be due to one of its many constituents other than shogaol.

The Applicant has thus discovered, surprisingly, that shogaol(s), (a) compound(s) of the arylalkanone family, has(have) an aphrodisiac action on men and/or women.

The present invention thus relates to the use of one or more shogaol(s) as aphrodisiacs.

For the purposes of the present invention, the term “aphrodisiac” means the stimulation or arousal of the libido, i.e. the sexual desire.

Advantageously, the present invention relates to the use of one or more shogaol(s) as erectogenic agents or to stimulate or arouse the libido in humans.

Advantageously, the shogaol(s) correspond(s) to the general formula (I): 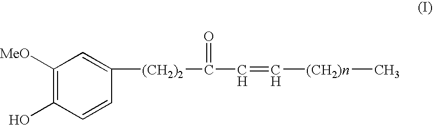
in which n is equal to 1, 2, 4, 6 or 8, and which are known, respectively, as [3]-shogaol, [4]-shogaol, [6]-shogaol, [8]-shogaol and [10]-shogaol. Even more advantageously, it is [3]-shogaol.

According to one particular embodiment, the shogaol(s) is(are) in the form of a crude extract of a plant of the Zingiberacea family, advantageously obtained by a process that comprises the following step:
a) preparation of a crude extract from fresh or dry rhizomes of the said plant, by macerating a ground material of these rhizomes at a temperature of between 10 and 35° C., followed by one or more extractions at reflux of this ground material, or by subjecting a ground material of the said rhizomes to a percolation at a temperature of between 10 and 35° C., each of these operations (maceration, extractions at reflux and percolation) being performed by means of a suitable organic solvent or mixture of organic solvents.

The maceration of the ground rhizome material, prior to its extraction, mainly has the effect of improving the placing in contact of the plant tissues and of the solvent during the extraction. Its duration may be between about twelve hours and one week depending on the freshness of the rhizomes used.

For the maceration, extraction at reflux and percolation operations on the ground material, water-miscible organic solvents with a relatively low boiling point are advantageously used so as to be able subsequently to remove them easily by a simple evaporation, such as ethanol, methanol or acetone or mixtures thereof with water. However, since the shogaols are soluble in many organic solvents, it is also possible to use other organic solvents, such as ethyl acetate, ethyl ether, chloroform or methylene chloride.

In another particular embodiment, the shogaol(s) is(are) in the form of a purified extract of a plant of the Zingiberacea family, advantageously obtained by a process that comprises, besides step a) described above, the following additional steps:
b) purification of the crude extract obtained in step a) by subjecting the said extract, after removing the solvent(s) it contains and taking it up in water, to one or more counter-current extractions with a water-immiscible organic solvent or mixture of organic solvents, and, if desired,
c) chromatographic separation of the shogaols.

The water-immiscible organic solvent(s) that are useful for carrying out the counter-current extractions of the crude extract in order to purify it are those chosen especially from ethyl acetate, ethyl ether, chloroform and methylene chloride, and mixtures thereof.

Advantageously, the plant of the Zingiberacea family is chosen from the species alpinia galanga, alpinia officinarum, Zingiber officinalis, Zingiber cassumunar and Zingiber zerumbet, and even more advantageously it is Alpinia galanga.

For example, [3]-shogaol, [6]-shogaol and [8]-shogaol may be extracted from plants of the Alpinia genus such as Alpinia galanga or Alpinia officinarum, whereas [4]-shogaol and [10]-shogaol may be extracted from plants of the Zingiber genus such as Zingiber officinalis, Zingiber cassumunar or Zingiber zerumbet, in particular using the process as described above.

Advantageously, the crude extract of Alpinia galanga contains an amount of [3]-shogaol of between, by weight, about 1 to 5% of the dry weight of the said extract. In accordance with the invention, this extract is obtained from fresh or dry rhizomes of the said plant.

Advantageously, the purified extract of Alpinia galanga contains an amount of [3]-shogaol at least equal by weight to 75% of the dry weight of the said extract.

The present invention also relates to the use of one or more shogaol(s) for the preparation of an aphrodisiac composition.

Advantageously, the aphrodisiac composition is formulated for an oral administration, for example, in the form of powders, drinkable solutions or suspensions, syrups, tablets or gel capsules.

The present invention also relates to a process for arousing or stimulating the libido in human beings, characterized in that it comprises the administration to a human being of an effective amount of one or more shogaol(s) as defined above.

The examples that follow for the preparation of extracts containing shogaols are given purely as illustrations of the subject of the invention, of which they do not in any way constitute a limitation.

### EXAMPLE 1

### Preparation of a Crude Extract of Rhizomes of Alpinia galanga

One kilo of fresh rhizomes of Alpinia galanga is ground coarsely, taking care not to overheat the ground portions. The water content of the ground material thus obtained is determined, and it is left to macerate in 7 litres of ethanol, the titre of which is selected such that, given the water content of the ground material, the maceration solvent is 50% ethanol.

After macerating for 24 hours at about 20° C., the ground material is extracted at reflux with the maceration solvent for 30 minutes. This solvent is removed and replaced with an equal weight of 50% ethanol, and the ground material is extracted again at reflux for 30 minutes. The operation is repeated once.

The 3 extracts obtained are combined (thus constituting a volume of about 19 litres), filtered on paper and then evaporated to dryness under reduced pressure.

A residue which weighs about 50 g is obtained, i.e. an approximate yield of 30% relative to the dry weight of the rhizomes. This extract contains the various shogaols present in the Alpinia galanga rhizomes ([3]-shogaol, [6]-shogaol and [8]-shogaol) and its content of [3]-shogaol is generally between 1 and 5% (w/w) according to the rhizomes used.

### EXAMPLE 2

### Preparation of a Purified Extract of Alpinia galanga Rhizomes

50 g of a crude extract, prepared in accordance with Example 1, are taken up in 1 litre of distilled water and the mixture is boiled for 1 minute with constant stirring. Stirring is continued until this extract is completely homogenized, and it is then cooled. It is then subjected to 4 successive counter-current extractions each with 100 ml of ethyl ether.

The ether solutions are combined; anhydrous sodium sulphate is added thereto to remove the water they contain; they are filtered through paper and evaporated to dryness under reduced pressure.

A residue that weighs 6.8 g is thus obtained, i.e. a yield of about 4% relative to the dry weight of the rhizomes. This extract, which mainly contains [3]-shogaol, has a [3]-shogaol content that is generally greater than 75% (w/w)

### EXAMPLE 3

### Production of [3]-shogaol

[3]-Shogaol may be obtained from Alpinia galanga rhizomes by preparing a crude extract of these rhizomes in accordance with Example 1, then purifying this extract in accordance with Example 2, and then subjecting the extract thus purified to successive elutions on columns of silica gel, for example in the following manner.

10 g of a purified extract, prepared in accordance with Example 2, are supplemented with 100 g of a silica gel G60 and 500 ml of chloroform with constant stirring. As soon as the mixture is homogeneous, it is evaporated to dryness under reduced pressure so as to obtain a powder.

This powder is placed at the top of a column 10 cm in diameter and 50 cm long, also containing silica gel G60 in petroleum ether. The column is first eluted with petroleum ether until the residue is less than 0.1% (about 10 litres of petroleum ether required to achieve this stage), then with 12 litres of benzene and finally with 8 litres of chloroform.

The chloroform phase is evaporated to dryness under reduced pressure, leaving a residue of about 2.3 g. This residue is then subjected to a preparative chromatography on a column 5 cm in diameter and 20 cm in length, filled with C18 silica gel, and using a water/acetonitrile mixture (70/30) as elution gradient. The fraction containing the [3]-shogaol is eluted in a time of between 5 and 7 minutes for a flow rate of 30 ml/min.

The [3]-shogaol may be identified by high pressure liquid chromatography (HPLC) coupled to a mass spectrometer.

### EXAMPLE 4

### Preparation of an Aphrodisiac Composition in Oral Form

1 kilo of crude extract, prepared in accordance with Example 1, was mixed intimately with 1 kilo of maltodextrin in a knife mill so as to ensure better homogeneity of the mixture and to obtain a non-sticky mobile powder. This powder was then distributed in No. 0 gel capsules so as to obtain a unit dose of 250 mg of crude extract.

Similarly, gel capsules may be manufactured using [3]-shogaol to obtain a unit dose of 25 mg of [3]-shogaol.

These crude or purified extracts or the shogaol(s) were tested successfully in oral form as regards their aphrodisiac action on men and women.

1-7. (canceled)

8. An aphrodisiac medicinal product, comprising at least one shogaol.

9. The product of claim 8, wherein the shogaol corresponds to the general formula (I): 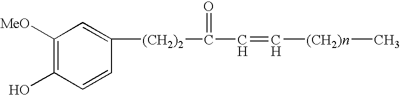
in which n is equal to 1, 2, 4, 6 or 8.

10. The product of claim 9, wherein n is equal to 1.

11. The product of claim 8, wherein the shogaol is in the form of a crude extract of a plant of the Zingiberacea family.

12. The product of claim 11, wherein the crude extract is obtained by a process comprising the following step:
a) preparing the crude extract from fresh or dry rhizomes of the plant by macerating a ground material of the rhizomes at a temperature of between 10 and 35° C., followed by extracting at least once the ground material at reflux, or by subjecting the ground material of the rhizomes to a percolation at a temperature of between 10 and 35° C., each of the maceration, extraction at reflux and percolation operations being performed by means of an effective organic solvent or a mixture of organic solvents.

13. The product of claim 12, wherein the crude extract is obtained by a process comprising step a) as defined in claim 12 and the following additional steps:
b) purifying the crude extract obtained in step a) by subjecting the extract, after removing the solvent it contains and taking it up in water, to at least one counter-current extraction with a water-immiscible organic solvent or mixture of water-immiscible organic solvents, and, optionally,
c) chromatographically separating the shogaol.

14. The product of claim 11, wherein the plant of the Zingiberacea family is the species Alpinia galanga, Alpinia officinarum, Zingiber officinalis, Zingiber cassumunar or Zingiber zerumbet.

15. The product of claim 14, wherein the plant of the Zingiberacea family is Alpinia galanga.

16. The product of claim 8, wherein the product is an erectogenic medicinal product or a medicinal product for arousing or stimulating the libido in a human.

17. The product of claim 8, wherein the product is formulated for oral administration.

18. A process for arousing or stimulating the libido in a human, comprising administering to the human an effective amount of at least one shogaol.

19. The process of claim 18, wherein the arousal or stimulation of the libido is erectogenic.
